(19) United States
(12) Patent Application Publication
(10) Pub. No.: US 2025/0057983 A1
(43) Pub. Date: Feb. 20, 2025

(54) COMPOSITION FOR PROMOTING MIGRATION OF ADULT STEM CELLS USING INCREASE IN EXPRESSION OF MIRNA

(71) Applicant: Soonchunhyang University Industry Academy Cooperation Foundation, Chungcheongnam-do (KR)

(72) Inventors: Nayoung Suh, Seoul (KR); Dayeon Kang, Jeju-do (KR)

(73) Assignee: Soonchunhyang University Industry Academy Cooperation Foundation, Chungcheongnam-do (KR)

(21) Appl. No.: 18/791,283

(22) Filed: Jul. 31, 2024

(30) Foreign Application Priority Data
Aug. 1, 2023 (KR) 10-2023-0100390

(51) Int. Cl. A61K 48/00; C12N 15/113

(52) U.S. Cl. CPC A61K 48/0058; C12N 15/113; C12N 2310/141

(57) ABSTRACT

The present invention relates to a composition for promoting the migration of adult stem cells, including miR-29a or miR-30c as an active ingredient, miR-29a or miR-30c, and a method for promoting the migration of adult stem cells, and it has been confirmed that the overexpression of miR-29a or miR-30c restores polarity, forms normal focal adhesions, and restores actomyosin-dependent contractile and traction forces, thereby promoting the migration of stem cells.

### CROSS-REFERENCE TO RELATED APPLICATION

This application claims priority to and the benefit of Korean Patent Application No. 2023-0100390, filed on Aug. 1, 2023, the disclosure of which is incorporated herein by reference in its entirety.

### BACKGROUND

### 1. Field of the Invention

The present invention relates to a composition for promoting the migration of adult stem cells in order to promote the migration, and a method for promoting the migration of adult stem cells.

### 2. Discussion of Related Art

Among the cells which are present in our bodies, cells that have the ability to differentiate into various types of cells are referred to as “stem cells.” Adult stem cells are stem cells which are present in grown adults, that is, in human blood, fat, bone marrow, nerves, muscles, skin, umbilical cord blood, placenta, and the like, and examples of adult stem cells include hematopoietic stem cells, mesenchymal stem cells, neural stem cells, and the like. Among them, mesenchymal stem cells are multipotent stromal cells (cells which surround and support the cells or tissues (parenchyma) which perform their functions in organs such as the thymus and bone marrow) and may differentiate into various cells including osteoblasts (bone cells), chondrocytes, myocytes, and adipocytes (adipocytes that produce bone marrow adipose tissue) (Ankrum et al., 2014).

Mesenchymal stem cells may be isolated from various tissues such as bone marrow, blood, umbilical cord blood and fat, and can be mass-proliferated under in vitro culture conditions, and thus have the advantages of being able to be used as source cells for a cell therapeutic agent without ethical issues. Further, they have the tropism to migrate to damaged tissues and sites of inflammation, thereby aiding in wound healing, and exhibit immunosuppressive properties under various conditions.

Meanwhile, mesenchymal stem cells have a weaker ability to migrate to damaged tissues and sites of inflammation than blood cells such as white blood cells. Although mesenchymal stem cells have the tropism to migrate to damaged tissues and sites of inflammation, the adhesion ligands and chemokine receptors required for this purpose are expressed in relatively small amounts, and it has been reported that even adhesion ligands and chemokine receptors are gradually lost during in vitro culture for mass proliferation (Rombouts et al., 2003). Although many studies have been conducted to induce the high expression of adhesion ligands and chemokine receptors, most of the studies have a disadvantage in that it is difficult to apply a viral system to clinical trials in humans using the viral system as a vector to efficiently deliver the corresponding genes.

Meanwhile, microRNA (miRNA) is a new substance that binds to the 3′-UTR of mRNA to control gene expression in eukaryotes as a single-stranded RNA molecule of approximately 21 to 25 nucleotides (nt). In the production of miRNA, a pre-miRNA of a stemloop structure is made by DGCR8/Drosha (RNaseIII type enzyme), which migrates to the cytoplasm, and is cleaved by a dicer to make miRNA. The miRNA thus produced is involved in development, cell proliferation and death, fat metabolism, tumorigenesis, and the like by regulating the expression of a target gene. Although miRNA having such functions has recently attracted attention as epigenetic regulators other than histone deacetylases (HDACs) or DNA methyl transferases (DNMTs), it has not been clear to date how the miRNA affects the migration of adult stem cells.

### SUMMARY OF THE INVENTION

The present invention is directed to providing a composition for promoting the migration of adult stem cells, including hsa-miR-29a (miR-29a) and/or hsa-miR-30c (miR-30c).

The present invention is also directed to providing a method for promoting the migration of adult stem cells by increasing the content of miR-29a and miR-30c in adult stem cells.

According to an aspect of the present invention, there is provided a composition for promoting the migration of adult stem cells, including, as an active ingredient, one or more selected from the group consisting of the nucleic acid molecules miR-29a and miR-30c.

According to another aspect of the present invention, there is provided a method for promoting the migration of adult stem cells, the method including increasing the content of one or more nucleic acid molecules selected from the group consisting of the nucleic acid molecules miR-29a and miR-30c in adult stem cells.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIGS. 1A-D show that a miRNA-29a mimic and/or a miRNA-30C mimic enhanced the migration of stem cells lacking expression of a gene associated with DiGeorge syndrome chromosomal region 8 (DGCR8). FIG. 1A shows bright field images of the results of a transwell migration assay confirming the migration of stem cells in vitro; FIG. 1B shows the quantification of the migration assay results; and FIG. 1C and FIG. 1D show schematic views of the experimental schedule.

FIGS. 2A-D show that miR-29 enhanced the migration of adult stem cells toward stromal cell-derived factor 1 (SDF-1α). FIG. 2A and FIG. 2B show bright field images of the results of a transwell migration assay confirming the migration of stem cells in vitro toward SDF-1α; FIG. 2C shows the quantification of the migration assay results; and FIG. 2D shows a schematic view of the experimental schedule.

FIGS. 3A-E show that miR-29a enhanced the migration of adult stem cells in vivo. FIG. 3A shows a schematic of an in vivo migration assay in immunocompromised mice; FIGS. 3B and 3D show the fluorescence intensity of control (FIG. 3B) and miR-29a (FIG. 3D)-treated stem cells in vivo; FIGS. 3C and 3E show the quantification of the results of the in vivo migration assay based on the distance traveled, confirming that miR-29a enhanced the migration of stem cells in vivo.

FIGS. 4A-D show that miR-29a treatment restored stem cell polarity in stem cells lacking DGCR8 expression. FIG. 4A shows an immunofluorescence staining illustrating the polarity of control stem cells; FIG. 4B shows an immunofluorescence staining demonstrating that siDGCR8 treatment induced abnormal cell polarity; and FIG. 4C shows an immunofluorescence staining confirming that the abnormal polarity of siDGCR8-treated stem cells was restored with miR-29a treatment. FIG. 4D and FIG. 4E show western blot results confirming the enhanced expression of Rock1/2 (FIG. 4D) and myosin light chain 2 (MLC2) and phosphorylated MLC2 (FIG. 4E), these proteins are involved in the formation of cell polarization.

FIGS. 5A-G show that miR-29a treatment normalized focal adhesion formation and maturation in siDGCR8-treated stem cells. FIGS. 5A-C show immunofluorescent staining of a focal adhesion protein paxillin in stem cells treated with control siRNA (FIG. 5A); siDGCR8 (FIG. 5B), and siDGCR8 and miR-29a (FIG. 5C), which confirmed that miR-29a normalized a focal adhesion formation and maturation stage of stem cells in stem cells lacking the expression of DGCR8. FIGS. 5D-G show graphs quantifying the absolute number of focal adhesions per cell (FIG. 5D), size of focal adhesions per cell (FIG. 5E); focal adhesion morphology characterized by the proportion of enlarged focal adhesions per cell (FIG. 5F) and focal adhesion aspect ratio (FIG. 5G).

FIGS. 6A-G show that miR-29a treatment restored actomyosin-dependent contractile force in siDGCR8-treated stem cells. FIGS. 6A-C shows immunofluorescence images of myosin and F-actin, which are components of actomyosin, in stem cells treated with control siRNA (FIG. 6A), siDGCR8 (FIG. 6B), and siDGCR8 and miR-29a (FIG. 6C), and confirm the restoration of actomyosin-dependent contractile force in stem cells by miR-29a. FIGS. 6D-F shows graphs quantifying the intracellular location and expression level of F-actin and myosin in stem cells treated with control siRNA (FIG. 6D), siDGCR8 (FIG. 6E), and siDGCR8 and miR-29a (FIG. 6F). FIG. 6G shows a graph showing the correlation of interaction between actin and myosin in stem cells treated with control siRNA, siDGCR8, and siDGCR8 and miR-29a.

FIGS. 7A-B show that miR-29a treatment regulated stem cell traction force during migration. FIG. 7A shows bright field (BF) images, fluorescent beads images using cell traction force microscopy and the contractile force stress map of stem cells treated with control siRNA (stress map: 0-396), siDGCR8 (stress map: 0-110), and siDGCR8 and miR-29a (stress map: 0-297), confirming the regulation of stem cell traction force by miR-29a. FIG. 7B shows a graph quantifying the traction force of stem cells treated with control siRNA, siDGCR8, and siDGCR8 and miR-29a during migration.

FIGS. 8A-F show that miR-29a treatment regulated golgi and actin polarization and migration using a wound healing assay. FIG. 8A shows bright field images of stem cells treated with control siRNA, siDGCR8, and siDGCR8 and miR-29a at the beginning (0 h) and the end (8 h) of the wound healing assay, and illustrates the enhanced closure of the wound area by miR-29a treatment; and 8B shows a graph quantifying the results of FIG. 8A. FIG. 8C shows bright field images illustrating the polarization of the golgi apparatus in stem cells treated with control siRNA, siDGCR8, and siDGCR8 and miR-29a using GM130 staining and confirming the restoration of the Golgi polarization by miR-29a treatment FIG. 8D shows a graph quantifying the results of FIG. 8C. FIG. 8E shows bright field images illustrating the directionality of the actin filament in stem cells treated with control siRNA, siDGCR8, and siDGCR8 and miR-29a using rhodamine phalloidin and confirming that the direction of actin filaments was restored by miR-29a treatment and FIG. 8F shows a graph quantifying the results of FIG. 8E.

### DETAILED DESCRIPTION OF EXEMPLARY EMBODIMENTS

The present inventors have discovered a method for promoting the migration of adult stem cells using miR-29a or miR-30c which is microRNA, thereby completing the present invention.

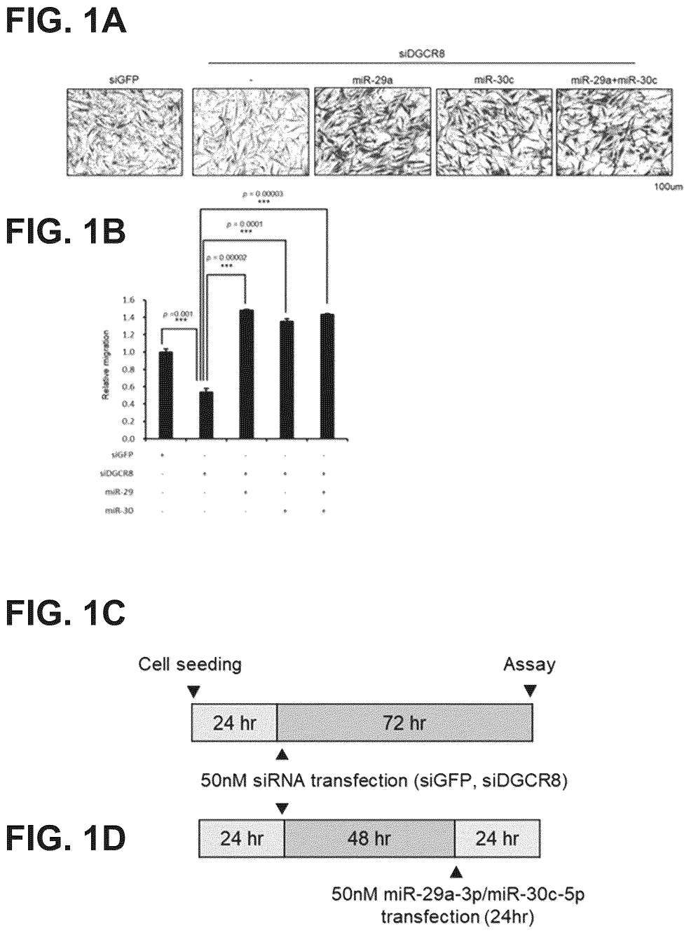

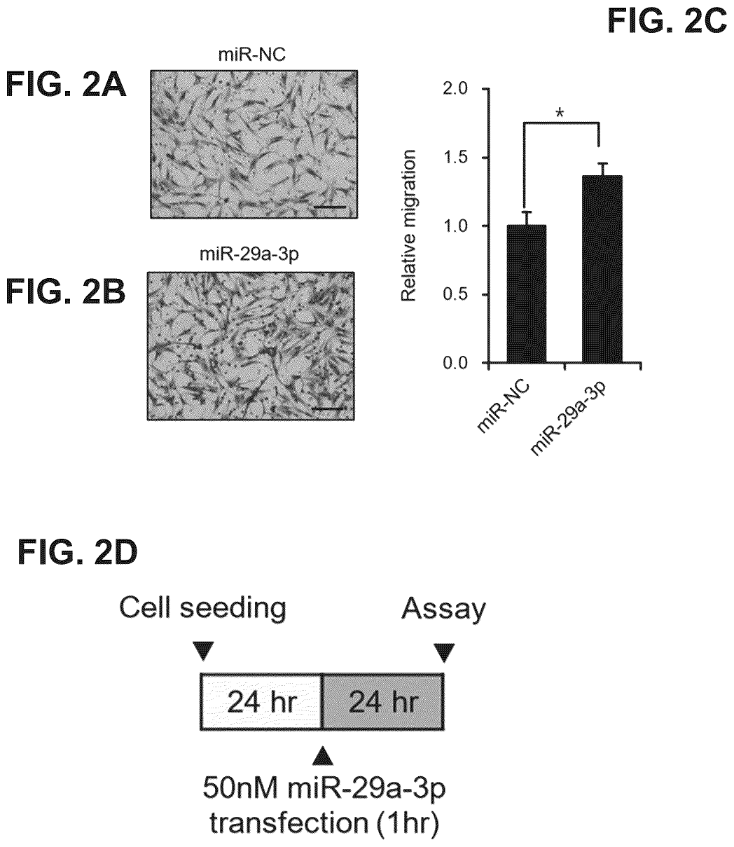

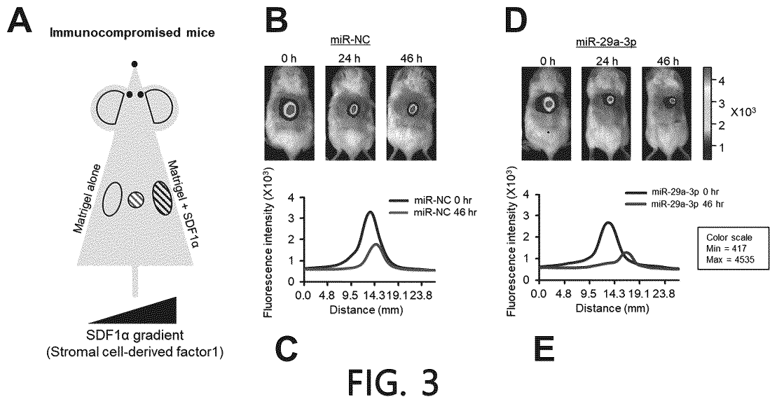

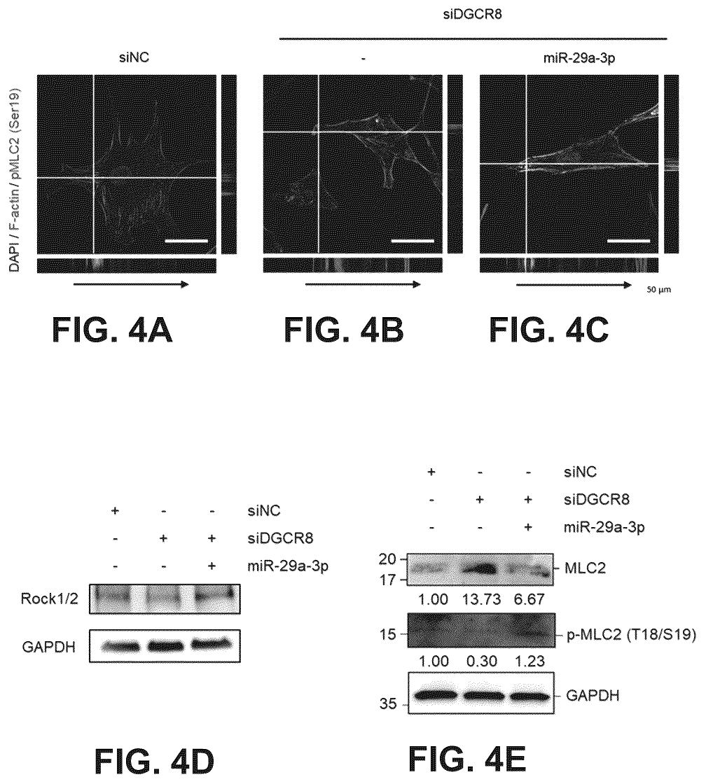

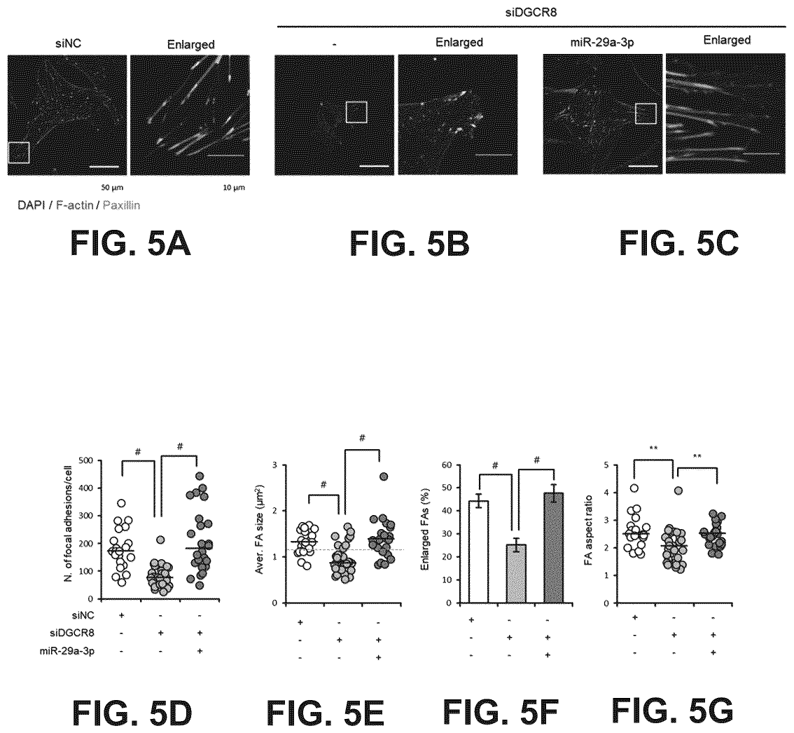

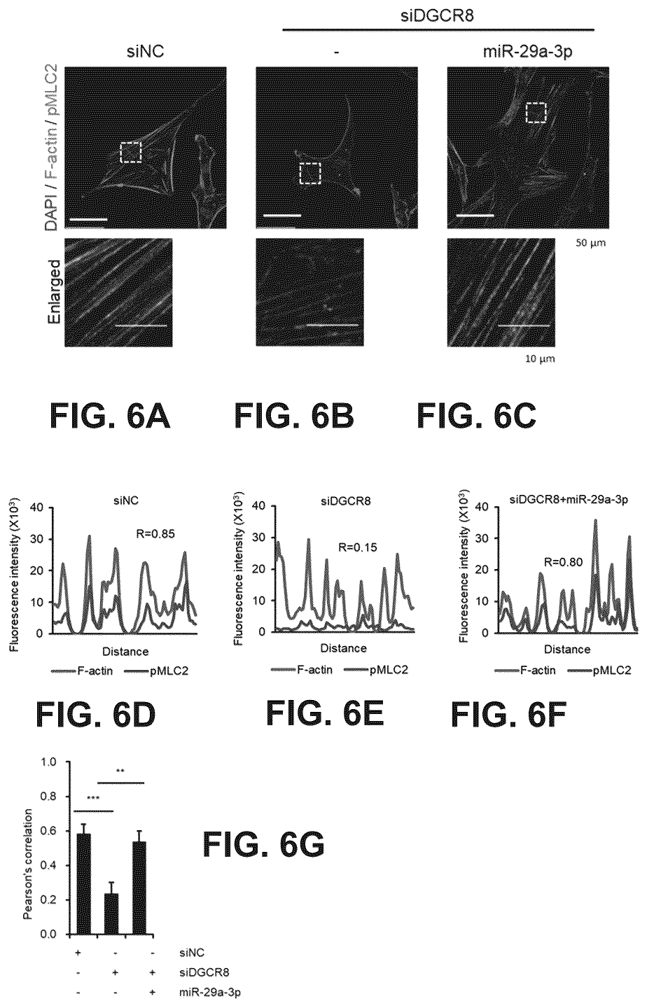

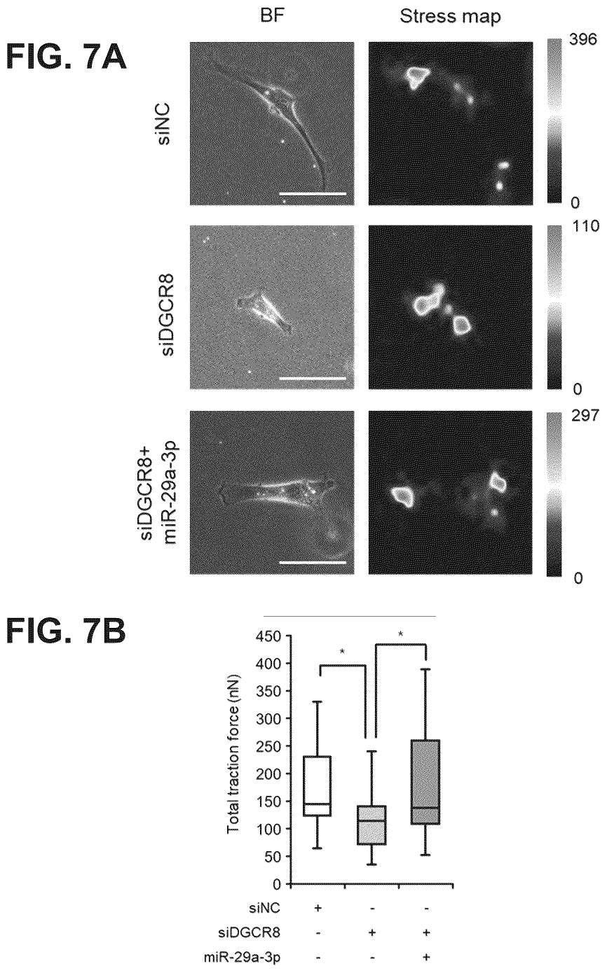

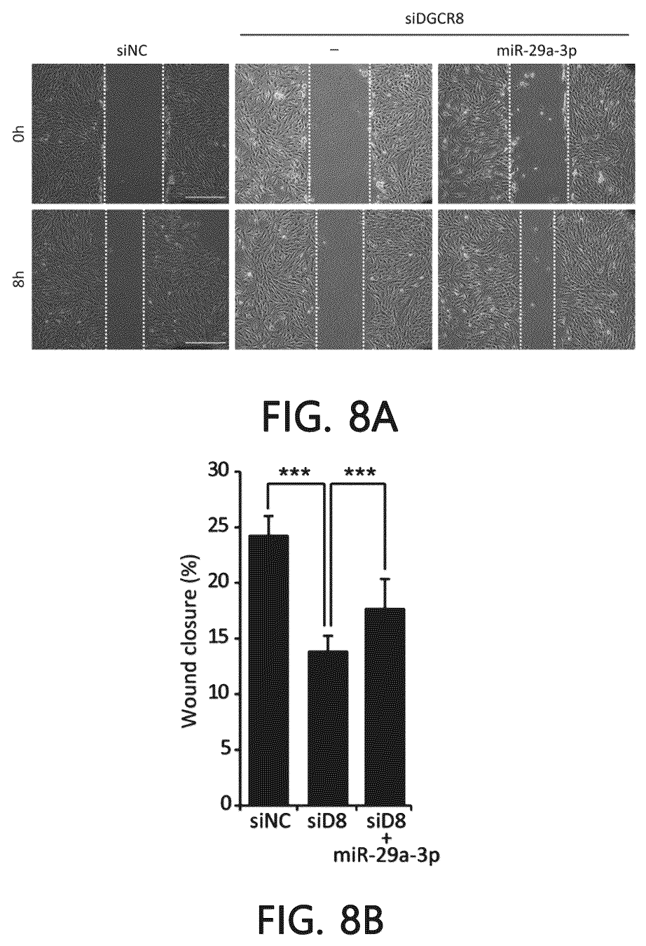

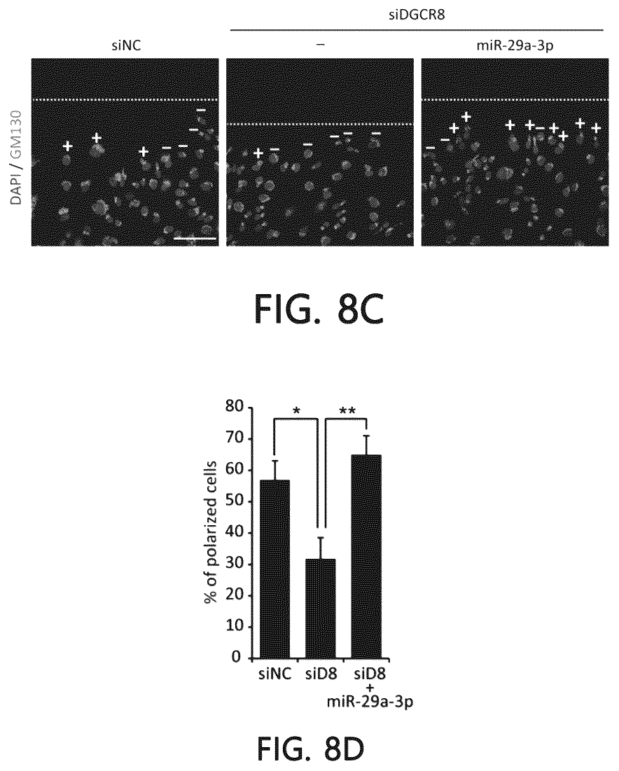

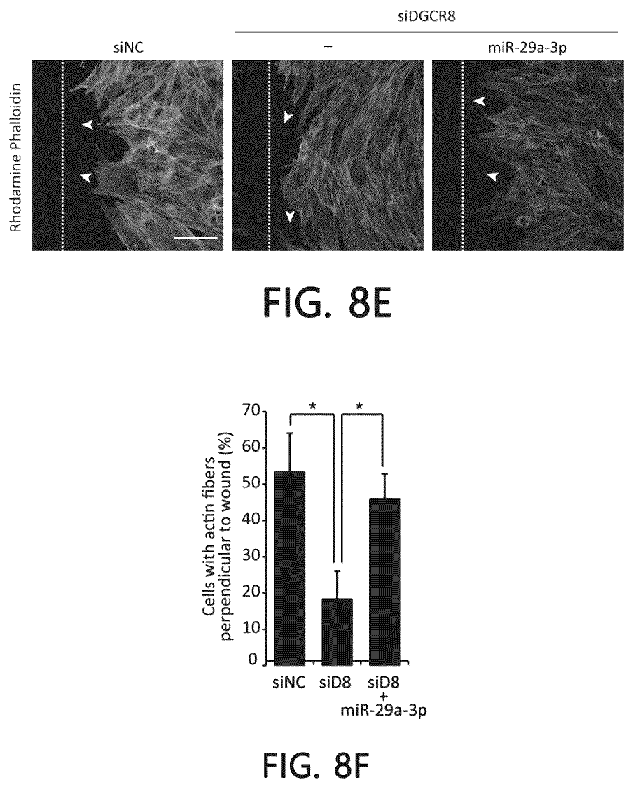

Specifically, in the present invention, it was confirmed that the migration of cells was measured using a transwell assay in cells in which DiGeorge syndrome chromosomal region 8 (DGCR), a gene essential for microRNA biogenesis, was silenced, and the addition of miR-29a and/or miR-30c increased the migration of stem cells compared to both a group in which miRNA expression was suppressed and a control (FIGS. 1A and 1B). Furthermore, it was confirmed using a transwell assay that when miR-29a was added to wild-type stem cells (FIGS. 2B and 2C), the migration toward stromal cell-derived factor 1 (SDF-1α), which is known to be secreted from the damaged site of cells, was increased compared to the control (FIGS. 2A-2C). Further, it was confirmed that even in mouse experiments using immunodeficient mice (FIG. 3A), the migration of stem cells into which miR-29a had been introduced was increased toward SDF-1α (FIGS. 3D and 3E) compared to the control (FIGS. 3B and 3C). In addition, as a result of confirming which mechanism promotes the migration of stem cells, it was confirmed that in stem cells into which miR-29a had been introduced, by the overexpression of miR-29a, polarity was restored (FIGS. 4C-4E), normal focal adhesions were formed (FIGS. 5C-5G), actomyosin-dependent contractile force (FIGS. 6C, 6F, and 6G) and traction force (FIGS. 7A and 7B) were restored, and the Golgi was polarized and actin filaments were rearranged (FIGS. 8A, 8B, 8C, 8D, 8E and 8F) to promote the migration.

As an aspect, the present invention provides a composition for promoting the migration of adult stem cells, including, as an active ingredient, one or more selected from the group consisting of the nucleic acid molecules miR-29a and miR-30c.

Two types of mature miRNAs are generated from a precursor, but when they are derived from different arms (3′ arm or 5′ arm) of the same pre-microRNA, they are named miR-n-3p or miR-n-5p by adding ‘-3p’ or ‘-5p’ to the rear side. In this case, n is a number, generally indicating the order of naming. Furthermore, miRNAs with almost the same sequence except for one or two sequence(s) are named by adding lowercase letters, and for example, miR-121a and miR-121b are generated from their respective precursors mir-121a and mir-121b, and have very similar sequences. In this case, “mir-” refers to the pre-miRNA, and “miR-” with a capital letter refers to the mature miRNA. Further, the species-specific naming of miRNAs is written first; for example, hsa-miR-123 is a human (Homo sapiens) miRNA, and oar-miR-123 is a sheep (Ovis aries) miRNA.

The precise names of miR-29a and miR-30c used in an exemplary embodiment of the present invention are hsa-miR-29a-3p and hsa-miR-30c-5p, which are mature miRNAs generated from their precursors hsa-miR29a and hsa-miR-30c, respectively. The sequences of the miR-29a and miR-30c are as follows: miR-29a, UAGCACCAUCUGAAAUCGGUUA (SEQ ID NO: 1); miR-30c, UGUAAACAUCCUACACUCUCAGC (SEQ ID NO: 2).

Currently, there are many established nomenclature systems for miRNAs, but before they were established, different researchers used slightly different nomenclature systems for miRNAs. Therefore, it is most accurate and desirable to track miRNAs by their accession numbers, which are unique numbers for miRNAs. The accession numbers of miR-29a and miR-30c used in an exemplary embodiment of the present invention are as follows: miR-29a, MIMAT0000086; miR-30c, MIMAT0000244.

Further, the adult stem cells mentioned in the present specification may be human adult stem cells or animal adult stem cells. Specifically, the adult stem cells may be mesenchymal stem cells (MSCs), multipotent stem cells, or amniotic epithelial cells. In addition, the mesenchymal stem cells may be derived from a source selected from the group consisting of fat, umbilical cord, umbilical cord blood, bone marrow, muscles, nerves, skin, amniotic membrane, and placenta. The method for obtaining stem cells from each source may be any method known in the related art, and is not limited to the method described in the examples of the present invention.

The miRNA nucleic acid molecule miR-29a or miR-30c of the present invention may be used to promote the migration of adult stem cells. As used herein, “migration” refers to the migratory activity of therapeutic cells to efficiently migrate from a transplant site to a damaged site.

Furthermore, as an aspect, the present invention provides a method for promoting the migration of adult stem cells by increasing the content of miRNA, which is miR-29a or miR-30c, in adult stem cells.

In the present invention, the content of the miRNA is increased by a method of introducing the miRNA into adult stem cells, a method of increasing the copy number of a gene encoding the miRNA in cells and introducing a mutation into the expression regulatory sequence of a gene on a chromosome encoding the miRNA, a method of replacing the expression regulatory sequence of a gene on a chromosome encoding the miRNA with a sequence with a stronger activity, a method of replacing a gene encoding the miRNA on a chromosome with a gene mutated to increase the activity of the miRNA, a method of introducing a mutation into a gene on a chromosome encoding the miRNA to enhance the activity of the miRNA, and the like, and the method is not limited thereto. Preferably, the content of miRNA may be increased by a transfection method, and here, transfection refers to a method of directly introducing miRNA into cultured animal cells to temporarily mutate the genetic traits of the cells, and may be performed by various methods known in the art, such as calcium phosphate-DNA coprecipitation, DEAE-dextran-mediated transfection, polybrene-mediated transfection, electric shock, microinjection, liposome fusion, lipofectamine, and circular solid fusion, but the method is not limited thereto.

Hereinafter, the configuration and effects of the present invention will be described in more detail through examples. These examples are only for illustrating the present invention, and the scope of the present invention is not limited by these examples.

### EXAMPLES

### Example 1: Method for Isolating and Culturing Human Mesenchymal Stem Cells

Human mesenchymal stem cells were isolated from adipose tissue of four donors aged between 33 and 46 years. The human mesenchymal stem cells were obtained, and then cultured in Dulbecco's modified Eagle's medium (Gibco) containing 10% fetal bovine serum (Gibco), 100 units/ml penicillin, and 100 μg/ml streptomycin (Gibco) in a humidified atmosphere containing 5% CO₂ at 37° C. The human mesenchymal stem cells were used in 3 to 7 passages.

### Example 2: Method for Culturing Immortalized Human Mesenchymal Stem Cells

Immortalized human mesenchymal stem cells (SCRC-4000, ATCC) are stem cells isolated from human adipose tissue, which overexpress human telomerase reverse transcriptase (hTERT). The immortalized human mesenchymal stem cells were cultured in Dulbecco's modified Eagle's medium (Gibco) containing 10% fetal bovine serum (Gibco), 100 units/ml penicillin, and 100 μg/ml streptomycin (Gibco) in a humidified atmosphere containing 5% CO₂ at 37° C.

### Example 3: Experimental Results

### Experimental Example 1: Ability of Adult Stem Cells to Migrate According to Treatment With miR-29a and/or miR-30c Using Transwell Assay

Transwells were used to evaluate the ability of stem cells to migrate. Transwell migration assays were performed using 24-well culture plates with inserts having an 8 μm pore size (Falcon). Human mesenchymal stem cells were proliferated to 50 to 60% in complete medium, and the next day, they were treated with siRNA against DGCR8 (DiGeorge syndrome chromosomal region 8, siDGCR8) or control (siGFP) at a concentration of 50 nM using Lipofectamine 2000 (Invitrogen) (FIG. 1C or 1D). Two days after siDGCR8 treatment, cells were treated with 50 nM miR-29a mimic (SEQ ID NO: 1) or miR-30c mimic (SEQ ID NO: 2) (Dharmacon) either separately or simultaneously for 24 hours using Dharmafect1 (Dharmacon; FIG. 1D). Thereafter, the treated mesenchymal stem cells were removed and prepared to include 3×10⁴ cells in 200 μl of serum-free medium. Stem cells prepared in the serum-free medium were seeded into the upper chamber of 8 μm pore cell culture inserts (Falcon), complete medium was placed in the lower chamber, and then, the stem cells were cultured under conditions of 5% CO₂ and 37° C. for 24 hours. Thereafter, the stem cells that had migrated to the lower side were fixed with 100% methanol and stained using 0.3% crystal violet. Thereafter, non-migrated cells were removed and the inserts were mounted on glass slides. Images were acquired using an Eclipse Ts2-FL inverted microscope (Nikon) (FIG. 1A), and cells were counted using ImageJ software (NIH) and normalized to the control (FIG. 1B).

It was confirmed that treatment with siDGCR8 reduced the migration of cells, but treatment with miR-29a and miR-30c, either individually or in combination, not only overcame the reduced mobility caused by siDGCR8, but also promoted migration more than the control (siGFP) (FIGS. 1A and 1B).

### Experimental Example 2: Measurement of Ability of Adult Stem Cells to Migrate Toward SDF-1α Using Transwell Assay

The ability of stem cells to migrate was confirmed from wild-type stem cells. Human mesenchymal stem cells were proliferated to 50 to 60% in complete medium, and one day later, they were treated with miR-29a mimic or a control (miR-NC; Dharmacon) at a concentration of 50 nM using Dharmafect1 (Dharmacon) for 1 hour, and the medium was replaced with fresh complete medium and used for the experiment the next day (FIG. 2D).

Thereafter, the treated mesenchymal stem cells were removed and prepared to include 3×10⁴ cells in 200 μl of complete medium. Stem cells prepared in the complete medium were seeded into the upper chamber of 8 μm pores cell culture inserts (Falcon), complete medium containing 100 ng/ml stromal cell-derived factor-1α (SDF-1α) was placed in the lower chamber, and then the stem cells were cultured under conditions of 5% CO2 and 37°° C. for 24 hours. Thereafter, the stem cells that had migrated to the lower side were fixed with 100% methanol and stained using 0.3% crystal violet. Thereafter, non-migrated cells were removed and the inserts were mounted on glass slides. Images were acquired using an Eclipse Ts2-FL inverted microscope (Nikon) (FIGS. 2A and 2B), and cells were counted using ImageJ software (NIH) and normalized to the control (FIG. 2C).

It was confirmed that treatment of wild-type stem cells with miR-29a increased the mobility of stem cells about 1.4-fold (FIGS. 2A, 2B, and 2C).

### Experimental Example 3: Measurement of Ability of Adult Stem Cells to Migrate In Vivo According to Treatment With miR-29a

To measure the ability of stem cells to migrate in vivo, a Matrigel plug assay was performed in immunodeficient mice. Six-week-old NOD/SCID male mice purchased from Orient Bio Inc. were used as the immunodeficient mice. In this case, Matrigel (Corning) containing 100 ng/ml SDF-1α was subcutaneously injected into the right side of the mouse back, and Matrigel alone was subcutaneously injected into the left side of the mouse back (FIG. 3A). The injected Matrigel is able to mimic the chemotaxis, in which cells migrate in vivo in response to differences in concentration of a chemical. Two hours after the injection of Matrigel, stem cells treated with the miR-29a mimic or control as described in Expermintal example 2 above were labeled with Qtracker™ 800 (Invitrogen) and injected subcutaneously into the center equidistant from the implant plug. As the cells, 106 cells were prepared in 50 μl of DPBS. Thereafter, images were captured at 0, 24, and 46 hours using an IVIS Spectrum In Vivo Imaging System (Perkin Elmer; FIGS. 3B and 3D). The captured images were analyzed using the ImageJ program (FIGS. 3C and 3E), and the results are shown in FIG. 3.

It was confirmed that when injected into mice, stem cells treated with miR-29a migrated further to the right over time compared to stem cells treated with the control (miR-NC) (FIGS. 3C and 3E).

### Experimental Example 4: Restoration of Stem Cell Polarity Establishment by Treatment With miR-29a

Since cells migrate through multiple steps involving regulation of cell polarization, protrusion, adhesion formation and maturation, traction force, and the like, it was confirmed through experiments how the migration mechanism of stem cells is changed by miR-29a treatment. First, to confirm the restoration of stem cell polarization, 3×10⁴ cells were cultured in Ibidi plates (Ibidi) under complete medium. The next day, 50 nM siDGCR8 and siNC (Dharmacon) were treated using Lipofectamine 2000 (Invitrogen). Two days after siDGCR8 treatment, the cells were treated with 50 nM miR-29a mimic (Dharmacon) using Dharmafect1 (Dharmacon). The next day, the cells treated with siRNA and miRNA were immobilized with 4% paraformaldehyde (PFA) for 15 minutes. After being washed with PBS for 15 minutes, the cells were treated with PBS supplemented with 0.1% triton X-100 and 1% BSA for 1 hour. Thereafter, the cells were treated with phospho-myosin light chain 2 (p-MLC2) (Thr18/Ser19) rabbit antibody (Cell Signaling Technology, 1:100) at 4° C. overnight. The next day, the cells were washed with PBS for 15 minutes and then treated with a mixture of rabbit Alexa Fluor 488 (Invitrogen, 1:2000) and rhodamine phalloidin (Invitrogen, 1:50) as secondary antibodies at room temperature for 2 hours. After being washed with PBS for 15 minutes, the cells were treated with 1 μg/ml DAPI (Sigma) diluted in PBS for 15 minutes. After being washed with PBS for 15 minutes, the cells were sealed with VECTASHIELD mounting solution (Vector Laboratories). Images were acquired using a THUNDER 215 Imager (Leica Microsystems Ltd.). The images were quantified using LAS X image-processing software 216 (Leica Microsystems Ltd.; FIGS. 4A-C) and ImageJ software (NIH).

It was confirmed that the abnormal polarity (FIG. 4A vs FIG. 4B) in transformed stem cells induced by siDGCR8 treatment (FIG. 4B) was restored by the overexpression of miR-29 a (FIG. 4C).

Further, western blot analysis was used to confirm the expression of proteins involved in the formation of cell polarization (FIGS. 4D-E). 6×10⁴ cells were cultured per well of a 6-well plate under complete medium. The next day, 50 nM siDGCR8 and siNC (Dharmacon) were treated using Lipofectamine 2000 (Invitrogen). Two days after siDGCR8 treatment, the cells were treated with 50 nM miR-29a mimic (Dharmacon) using Dharmafect1 (Dharmacon). The next day, cells were obtained and lysed in RIPA buffer (Biosesang) containing Halt protease and phosphatase inhibitor cocktail (Thermo Scientific). The cells were cultured on ice for 30 minutes while vortexing every 10 minutes, and then centrifuged at 12,000 rpm at 4° C. for 20 minutes, and the supernatant was obtained. Proteins were isolated using 4 to 20% Mini-PROTEAN TGX™ Precast Protein Gels (Bio-Rad, 185 Hercules, CA, USA) or 10% sodium dodecyl sulphate-polyacrylamide gel electrophoresis. Electrophoresed proteins were transferred to nitrocellulose membranes (GE Healthcare) and incubated in 1×TBST containing 1% or 5% BSA at room temperature for 1 hour. Thereafter, the membranes were incubated with appropriate primary antibodies at 4° C. overnight. In this case, the primary antibodies are as follows: ROCK 1+2 monoclonal rabbit antibody (Abcam, 1:1000), Myosin light chain 2 rabbit antibody (Cell Signaling Technology, 1:1000), Phospho-myosin light chain 2 (Thr18/Ser19) rabbit antibody (Cell Signaling Technology, 1:1000), GAPDH monoclonal mouse antibody (Santa Cruz Biotechnology, 1:1500). GAPDH was used as a loading control.

The membrane was washed with 1×TBST for 30 minutes and treated with the corresponding secondary antibody containing horseradish peroxide (HRP) at room temperature for 2 hours. In this case, the secondary antibodies are as follows: anti-mouse IgG, HRP-linked antibody (Cell Signaling Technology, 1:1000), anti-rabbit IgG, HRP-linked antibody (Cell Signaling Technology, 1:1000). After being washed with 1×TBST for 30 minutes, the membrane was visualized using Clarity Max Western ECL Substrate (Biorad), and images were obtained using a chemiluminescence imaging system c300 (Azure).

As shown in FIGS. 4D-4E, it was confirmed that the protein expression of Rock1/2 (FIG. 4D) and phosphorylated MLC2 (p-MLC2; FIG. 4D)), which are known to be involved in the formation of cell polarization, was increased by the overexpression of miR-29a.

### Experimental Example 5: Confirmation of Normalization of Focal Adhesion Formation and Maturation Stages in Stem Cells Treated With miR-29a

To confirm the focal adhesion of stem cells, 3×10⁴ cells were cultured in Ibidi plates (Ibidi) under complete medium. The next day, 50 nM siDGCR8 and siNC (Dharmacon) were treated using Lipofectamine 2000 (Invitrogen). Two days after siDGCR8 treatment, the cells were treated with 50 nM miR-29a mimic (Dharmacon) using Dharmafect1 (Dharmacon). The next day, the cells were immobilized with 4% paraformaldehyde for 15 minutes. After being washed with PBS for 15 minutes, the cells were treated with PBS supplemented with 0.1% triton X-100 and 1% BSA for 1 hour. Thereafter, the cells were treated with paxillin rabbit monoclonal antibody (Abcam, 1:100) at 4° C. overnight. The next day, the cells were washed with PBS for 15 minutes and then treated with a mixture of rabbit Alexa Fluor 488 (Invitrogen, 1:1000) and rhodamine phalloidin (Invitrogen, 1:50) as secondary antibodies at room temperature for 2 hours. After being washed with PBS for 15 minutes, the cells were treated with 1 μg/ml DAPI (Sigma) diluted in PBS for 15 minutes. After being washed with PBS for 15 minutes, the cells were sealed with VECTASHIELD mounting solution (Vector Laboratories). Images were obtained using a THUNDER 215 Imager (Leica Microsystems Ltd.), and the results are shown in FIGS. 5A-5C. The images were analyzed using LAS X image-processing software 216 (Leica Microsystems Ltd.) and ImageJ software (NIH) (FIGS. 5D-G).

It was confirmed that when mesenchymal stem cells were treated with siDGCR8, abnormal focal adhesions were shown (FIG. 5A vs FIG. 5B), but due to treatment with miR-29a, normal focal adhesions were formed and maturation was restored (FIG. 5A vs FIG. 5C). That is, it was confirmed that the abnormal intensity and distribution of paxillin and F-actin caused by SiDGCR8 was restored by miR-29a (FIG. 5A vs FIG. 5C). FIGS. 5D-5E show graphs quantifying focal adhesions, confirming that the absolute number (FIG. 5D) and size of focal adhesions (FIG. 5E) per cell were significantly reduced by treatment with SiDGCR8 and were restored by miR-29a. Further, focal adhesion morphology was characterized by quantifying the proportion of enlarged focal adhesions per cell (FIG. 5F) and their aspect ratio (FIG. 5G), and since the morphology was consistent with abnormal focal adhesions in siDGCR8-treated cells, it was confirmed that the percentages of enlarged focal adhesions per cell and their aspect ratio were significantly decreased and were restored by miR-29a.

### Experimental Example 6: Restoration of Actomyosin-Dependent Contractile Force of Stem Cells by Treatment With miR-29a

To confirm the actomyosin-dependent contractile force of stem cells, 3×10⁴ cells were cultured in Ibidi plates (Ibidi) under complete medium. The next day, 50 nM siDGCR8 and siNC (Dharmacon) were treated using Lipofectamine 2000 (Invitrogen). Two days after siDGCR8 treatment, the cells were treated with 50 nM miR-29a mimic (Dharmacon) using Dharmafect1 (Dharmacon). The next day, the cells were immobilized with 4% paraformaldehyde for 15 minutes. After being washed with PBS for 15 minutes, the cells were treated with PBS supplemented with 0.1% triton X-100 and 1% BSA for 1 hour. Thereafter, the cells were treated with phospho-myosin light chain 2 (Thr18/Ser19) rabbit antibody (Cell Signaling Technology, 1:100) at 4° C. overnight. The next day, the cells were washed with PBS for 15 minutes and then treated with a mixture of rabbit Alexa Fluor 488 (Invitrogen, 1:1000) and rhodamine phalloidin (Invitrogen, 1:50) as secondary antibodies at room temperature for 2 hours. After being washed with PBS for 15 minutes, the cells were treated with 1 μg/ml DAPI (Sigma) diluted in PBS for 15 minutes. After being washed with PBS for 15 minutes, the cells were sealed with VECTASHIELD mounting solution (Vector Laboratories). Images were acquired using a THUNDER 215 Imager (Leica Microsystems Ltd.; FIGS. 6A-6C). The images were quantified using LAS X image-processing software 216 (Leica Microsystems Ltd.) and ImageJ software (NIH) (FIGS. 6D-6F).

It was confirmed that the abnormal contractile force observed in stem cells transformed with siDGCR8 (FIGS. 6B and 6E) was restored by the overexpression of miR-29a (FIGS. 6C and 6F). It was confirmed that the correlation between actin and myosin was also restored by treatment with miR-29a (FIG. 6G).

### Experimental Example 7: Confirmation of Regulation of Stem Cell Traction Force by Treatment With miR-29a Using Cell Traction Force Microscopy

To perform cell traction force microscopy, collagen-conjugated polyacrylamide hydrogels loaded with 0.04% 0.5 μm fluorescent polystyrene microspheres (Invitrogen) were prepared. Polyacrylamide hydrogels were prepared on glass-bottom dishes (SPL) by mixing 5% acrylamide (Sigma), 0.225% bisacrylamide (Sigma), 1% ammonium persulfate (Sigma), and 0.1% tetramethyl ethylenediamine (TEMED; Sigma). Thereafter, the polyacrylamide gel was conjugated to 200 μg/ml rat tail collagen 1 (Corning) using sulfo-SANPAH (Thermo Scientific).

To confirm the change in stem cell traction force by miR-29a, 6×10⁴ cells per well of a 6-well plate were cultured under complete medium. The next day, 50 nM siDGCR8 and siNC (Dharmacon) were treated using Lipofectamine 2000 (Invitrogen). Two days after siDGCR8 treatment, the cells were treated with 50 nM miR-29a mimic (Dharmacon) using Dharmafect1 (Dharmacon). The next day, the cells were obtained to culture 1×10⁴ cells in the prepared dish under complete medium for 1 hour. After 1 hour, non-adherent cells were removed, and the medium was replaced with complete medium, and then the remaining cells were cultured in an incubator at 37° C. for 48 hours.

After 48 hours, the dishes were washed with DPBS and then transferred onto the stage of a fluorescence microscope. The cells were first photographed using bright field (FIG. 7A; BF) and fluorescent bead images (FIG. 7A, stress map), and then treated with 0.05% trypsin-EDTA (Gibco) for 15 minutes. Thereafter, the cells were completely dropped off using DPBS, and images of the migrated fluorescent beads were taken at the same position. The images were analyzed using template matching, particle image velocimetry (PIV), and Fourier transform traction cytometry (FTTC) ImageJ plugin (ImageJ plugin reference doi: 10.1016/bs.mcb.2014.10.008).

Cells exert traction forces on the bottom for migration and attachment. Therefore, when the cells fall, the traction force disappears. In this case, the traction force of the cells may be measured by analyzing the movement of the beads placed on the bottom. In FIGS. 7A and 7B, it was confirmed that the traction force reduced by siDGCR8 was restored by the overexpression of miR-29a.

### Experimental Example 8: Confirmation of Regulation of Cell Polarization and Migration During Wound Healing

To investigate how miR-29a-3p affects the stepwise process of stem cell migration at the cellular level, first, its role in cell polarization and cytoskeleton dynamics was confirmed using a wound healing assay (FIGS. 8A-8B). Stem cells transfected in the same manner as in Experimental Example 1 above were seeded into 24-well plates or u-Dishes 201 (ibidi) and grown for 24 hours. On the day of the experiment, the cells were scraped using a micropipette tip. For immunostaining, cells were allowed to be used for 4 hours before immobilization. Images were acquired using a Nikon Confocus AX (Nikon; FIG. 8A). Closure area, cell polarization, and actin orientation were evaluated using ImageJ software (NIH) (FIG. 8B).

It was confirmed that treatment with siDGCR8 reduced wound closure as measured by a wound healing assay, and this reduced migration was restored by miR-29a (FIGS. 8A and 8B).

Since the initial establishment of polarization and repositioning of the Golgi apparatus toward the frontal edge is essential for cell migration during wound healing, these were investigated by examining the polarized Golgi apparatus, labeled with GM130 staining, within a 120° sector toward the wound. To quantify cell polarization, migrating cells were immobilized with 4% paraformaldehyde and cultured in PBS containing 0.1% Triton X-100 and 1% BSA for 1 hour, and then reacted with GM130 rabbit antibody (12480, Cell Signaling Technology), rhodamine phalloidin (Invitrogen), and DAPI (Sigma). Images were obtained using a Nikon Confocal AX microscope (Nikon).

Unlike control cells, most of the DGCR8 knockdown cells in the front row were not polarized (FIG. 8C). However, the introduction of miR-29a reconstituted the Golgi apparatus. Specifically, 56.94% of siNC control cells showed polarized Golgi compared to 31.77% of DGCR8-knockdown cells, and 64.98% of miR-29a-overexpressing cells showed polarization (FIG. 8D). That is, it was confirmed that it is important for miR-29a to restore Golgi polarization in DGCR8-knockdown cells. Furthermore, 4 hours after wounding, actin filaments were organized perpendicular to the wound edge in both control and miR-29a-overexpressing stem cells (FIG. 8E). In contrast, actin filaments in DGCR8-knockdown cells were usually oriented parallel to the wound edge. The proportion of cells at the wound edge with a polarized distribution of F-actin was found to be 53.54% in control cells, 18.51% in DGCR8-knockdown cells, and 46.18% in miR-29a-overexpressing cells (FIG. 8F). That is, it was confirmed that miR-29a not only restores the Golgi polarization but also restores the proper orientation of actin filaments, which is essential for effective cell migration during wound healing.

The composition for promoting the migration of adult stem cells according to the present invention, including miR-29a and/or miR-30c as an active ingredient, can promote the migration of adult stem cells by forming normal polarity and focal adhesion and restoring actomyosin-dependent contractile and traction forces. Since this enables stem cells to migrate quickly and effectively to a target, it is expected to increase the therapeutic efficiency upon stem cell therapy.

What is claimed is:

1. A method for promoting the migration of adult stem cells, the method comprising increasing a content of one or more nucleic acid molecules selected from the group consisting of the nucleic acid molecules miR-29a and miR-30c in adult stem cells.

2. The method of claim 1, wherein the nucleic acid molecule miR-29a comprises the nucleotide sequence of SEQ ID NO:1, and the nucleic acid molecule miR-30c comprises the nucleotide sequence of SEQ ID NO:2.

3. The method of claim 1, wherein the content of the one or more nucleic acid molecules is increased by overexpression of the nucleic acid molecule miR-29a and/or miR-30c.

4. The method of claim 1, wherein the increasing the content of the nucleic acid molecules is performed by:
a) a method of introducing the nucleic acid molecule into adult stem cells;
b) a method of increasing a copy number of a gene encoding the nucleic acid molecules in cells;
c) a method of introducing a mutation into the expression regulatory sequence of a gene on a chromosome encoding the nucleic acid molecules;
d) a method of replacing the expression regulatory sequence of a gene on a chromosome encoding the nucleic acid molecules with a sequence with a stronger activity;
e) a method of replacing a gene encoding the nucleic acid molecules on a chromosome with a gene mutated to increase the activity of the nucleic acid molecules; or
f) a method of introducing a mutation into a gene on a chromosome encoding the nucleic acid molecules to enhance the activity of the nucleic acid molecules.

5. The method of claim 4, wherein the method of introducing the nucleic acid molecules into the adult stem cells is introducing the nucleic acid molecules into the adult stem cell by transfection.

6. The method of claim 1, wherein the method increases factors involved in a formation of polarization.

7. The method of claim 6, wherein the factors involved in the formation of polarization are Rock1/2 or phospho-Myosin Light Chain 2 (p-MLC2).

8. The method of claim 1, wherein the method increases one or more selected from the group consisting of the number, size, and aspect ratio of focal adhesion.

9. The method of claim 1, wherein the adult stem cells are mesenchymal stem cells.

10. The method of claim 9, wherein the mesenchymal stem cells are derived from any one selected from the group consisting of fat, umbilical cord, umbilical cord blood, bone marrow, muscles, nerves, skin, amniotic membrane, and placenta.

11. A method for healing a wound, the method comprising administering to a subject adult stem cells with increased content of one or more nucleic acid molecules selected from the group consisting of the nucleic acid molecules miR-29a and miR-30c.

12. The method of claim 11, wherein the nucleic acid molecule miR-29a comprises the nucleotide sequence of SEQ ID NO: 1, and the nucleic acid molecule miR-30c comprises the nucleotide sequence of SEQ ID NO:2.

13. The method of claim 11, wherein the adult stem cells are adult stem cells in which the nucleic acid molecules miR-29a and/or miR-30c are overexpressed.

14. The method of claim 11, wherein the adult stem cells with increased content of nucleic acid molecules are obtained by:
a) a method of introducing the nucleic acid molecule into adult stem cells;
b) a method of increasing a copy number of a gene encoding the nucleic acid molecule in cells;
c) a method of introducing a mutation into the expression regulatory sequence of a gene on a chromosome encoding the nucleic acid molecule;
d) a method of replacing the expression regulatory sequence of a gene on a chromosome encoding the nucleic acid molecule with a sequence with a stronger activity;
e) a method of replacing a gene encoding the nucleic acid molecule on a chromosome with a gene mutated to increase the activity of the nucleic acid molecule; or
f) a method of introducing a mutation into a gene on a chromosome encoding the nucleic acid molecule to enhance the activity of the nucleic acid molecule.

15. The method of claim 14, wherein the method of introducing the nucleic acid molecule into the adult stem cells is introducing the nucleic acid molecule into the adult stem cell by transfection.

16. The method of claim 11, wherein the method increases factors involved in a formation of polarization.

17. The method of claim 16, wherein the factors involved in the formation of polarization are Rock1/2 or phospho-Myosin Light Chain 2 (p-MLC2).

18. The method of claim 11, wherein the method increases one or more selected from the group consisting of the number, size, and aspect ratio of focal adhesion.

19. The method of claim 11, wherein the adult stem cells are mesenchymal stem

20. The method of claim 19, wherein the mesenchymal stem cells are derived from any one selected from the group consisting of fat, umbilical cord, umbilical cord blood, bone marrow, muscles, nerves, skin, amniotic membrane, and placenta.


SEQUENCE LISTING (2 sequences)

SEQ ID NO: 1 - RNA, 22 nt, synthetic construct
  source 1..22: mol_type = other RNA
tagcaccatctgaaatcggtta

SEQ ID NO: 2 - RNA, 23 nt, synthetic construct
  source 1..23: mol_type = other RNA
tgtaaacatcctacactctcagc